(19) United States
(12) Patent Application Publication
(10) Pub. No.: US 2008/0132870 A1
(43) Pub. Date: Jun. 5, 2008

(54) Combined disposable panty and sanitary pad undergarment

(76) Inventor: Edith Nelson, Andover, NJ (US)

Correspondence Address: JAMES RAY & ASSOCIATES, 2640 PITCAIRN ROAD, MONROEVILLE, PA, 15146, US

(21) Appl. No.: 11/999,363

(22) Filed: Dec. 5, 2007

Related U.S. Application Data
(60) Provisional application No. 60/868,594, filed on Dec. 5, 2006.

(51) Int. Cl. A61F 13/15

(52) U.S. Cl. 604/385.19; 604/385.24; 604/396; 604/385.01

(57) ABSTRACT

A combined disposable panty and sanitary pad undergarment includes a panty body having a front part, a rear part forming in combination with the front part a top edge of the panty body, and a crotch part joining the rear and front part and forming a pair of leg openings. An absorption pad is overlaid over and secured to the crotch part and a lower portion of each front and rear part. The absorption pad has a fluid-permeable human body facing layer, a fluid-impermeable panty body engaging layer and an absorbent layer disposed intermediate the fluid-permeable and the fluid-impermeable layers. A front end of the absorption pad is positioned above external genital organs of a wearer and a rear end thereof is positioned in close proximity to an anus. The side edges of the absorption pad are tapered to follow contour and style of the panty body.

### CROSS-REFERENCE TO RELATED APPLICATIONS

This application is related to and claims priority from Provisional Patent Application Ser. No. 60/868,594 filed on Dec. 5, 2006.

### FIELD OF THE INVENTION

The present invention relates, in general, to sanitary undergarments and, more particularly, this invention relates to a combined disposable panty and sanitary pad undergarment.

### BACKGROUND OF THE INVENTION

Managing periodic menstrual cycles, as is generally well known, requires the woman to ensure that the appropriate feminine products are always available for use and are correctly applied. Pads and tampons can be incorrectly placed and the optional wings or tabs on feminine pads may become crumpled, leading to ineffective coverage. Often, feminine pads offer little protection to every area of the panty body, which can lead to stains and ruined undergarments, thus requiring the repeated purchase of new panties. Many women prefer to have panties that are worn simply during menstrual cycles in order to prevent additional panties from being ruined. These panties normally contain ugly stains from a leaking pad or tampon. At other times, when one of the front or rear panel of the panty body is narrower than the other panel, the conventional pads and tampons protrudes beyond the boundary of the panty body and are not appealing to many wearers.

Prior to the conception and design of the present invention efforts have been made to alleviate disadvantages associated with leakage and staining from conventional sanitary pads. For example, U.S. Pat. No. 6,240,569 issued to Van Gompel et al. discloses a diaper-like disposable panty for holding a sanitary pad which is capable of trapping and absorbing the leakage from the pad and preventing liquid strike through onto outer clothing and bed linen. However, the diaper-like shape of the undergarment is unappealing to many wearing clinging or tight fitting clothing and is cumbersome to pull on/pull off.

Therefore, there is a need for an improved combined disposable panty and sanitary pad undergarment.

### SUMMARY OF THE INVENTION

The invention provides a combined disposable panty and sanitary pad undergarment. The undergarment includes a panty body. The panty body has a front part and a rear part which form, in combination, a top edge of the panty body. A crotch part joins the lower portions of the rear and front part and forms a pair of leg openings. An absorption pad is overlaid over and secured to the crotch part and the lower portion of each front and rear part. The absorption pad has a fluid-permeable human body facing layer, a fluid-impermeable panty body engaging layer and an absorbent layer disposed intermediate the fluid-permeable and the fluid-impermeable layers. The absorption pad has a predetermined length whereby a front end thereof is positioned above external genital organs of a wearer and whereby a rear end thereof is positioned in close proximity to an anus. It is also within the scope of the present invention to at least partially taper side edges of the absorption pad to follow contour and style of the panty body.

### OBJECTS OF THE INVENTION

It is, therefore, one of the primary objects of the present invention to provide a combined disposable panty and sanitary pad undergarment.

Another object of the present invention is to provide a combined disposable panty and sanitary pad undergarment that offers different levels of absorption.

Yet another object of the present invention is to provide a combined disposable panty and sanitary pad undergarment that includes side release devices.

A further object of the present invention is to provide a combined disposable panty and sanitary pad undergarment that can be manufactured in a variety of styles and sizes.

Yet a further object of the present invention is to provide a combined disposable panty and sanitary pad undergarment wherein the sanitary pad is contained within the boundary of the panty body and cannot shift or move.

An additional object of the present invention is to provide a combined disposable panty and sanitary pad undergarment that includes side edges thereof taper inwardly toward one end thereof.

Another object of the present invention is to provide a combined disposable panty and sanitary pad undergarment that includes a carrying case or a pouch formed from a material sufficient to prevent leakage or strike through from a soiled undergarment enclosed therewithin.

In addition to the several objects and advantages of the present invention which have been described with some degree of specificity above, various other objects and advantages of the invention will become more readily apparent to those persons who are skilled in the relevant art, particularly, when such description is taken in conjunction with the attached drawing Figures and with the appended claims.

### BRIEF DESCRIPTION OF THE DRAWINGS

FIG. 1 is a perspective view of a combined disposable panty and sanitary pad undergarment of the present invention;

FIG. 2 is a perspective view of the combined disposable panty and sanitary pad undergarment of FIG. 1, particularly illustrating the side release means;

FIGS. 3a-3f depict various styles of the combined disposable panty and sanitary pad undergarment of FIG. 1;

FIG. 4 is a cross-sectional view of the combined disposable panty and sanitary pad undergarment of FIG. 1; and

FIG. 5 depicts a storage or carrying pouch for use with the combined disposable panty and sanitary pad undergarment of FIG. 1.

### BRIEF DESCRIPTION OF THE VARIOUS EMBODIMENTS OF THE INVENTION

Prior to proceeding to the more detailed description of the present invention, it should be noted that, for the sake of clarity and understanding, identical components which have identical functions have been identified with identical reference numerals throughout the several views illustrated in the drawing figures.

The present invention overcomes the disadvantages of the prior art undergarments by providing a combined disposable panty and sanitary pad undergarment, generally designated as 10. Now in reference to FIG. 1, the undergarment 10 includes a one piece panty body 20 having a front part 22. A rear part 24 is also provided and forms in combination with the front part 22 a top edge 26 of the panty body 20. A crotch part 28 joins the lower portions of the rear and front parts 22, 24 and forms a pair of leg openings 30.

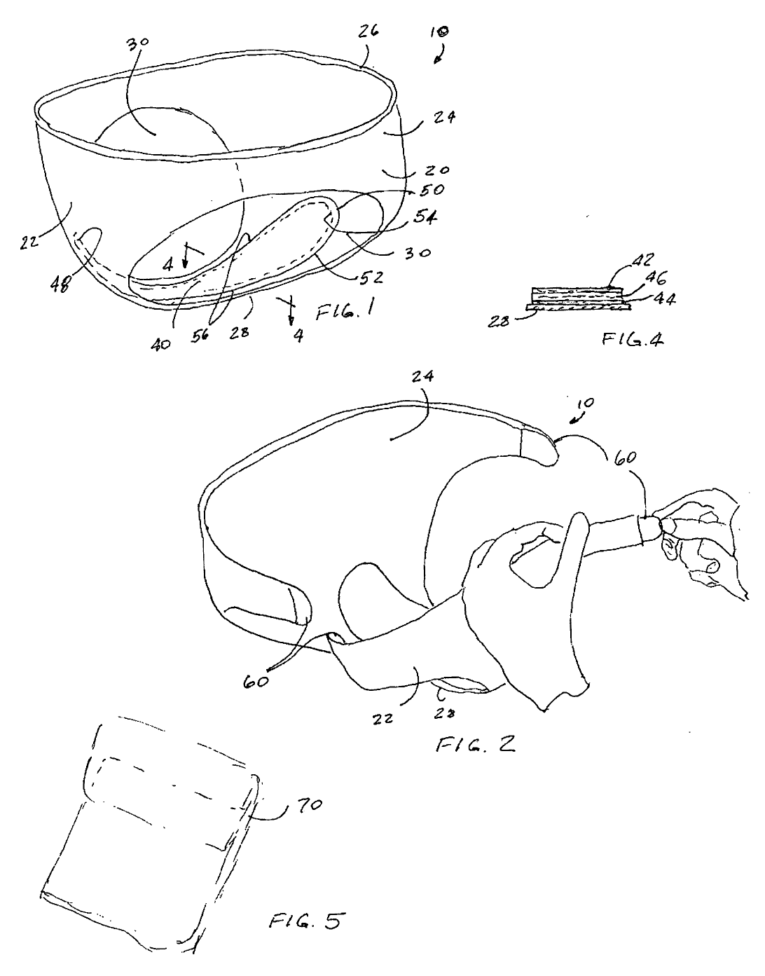

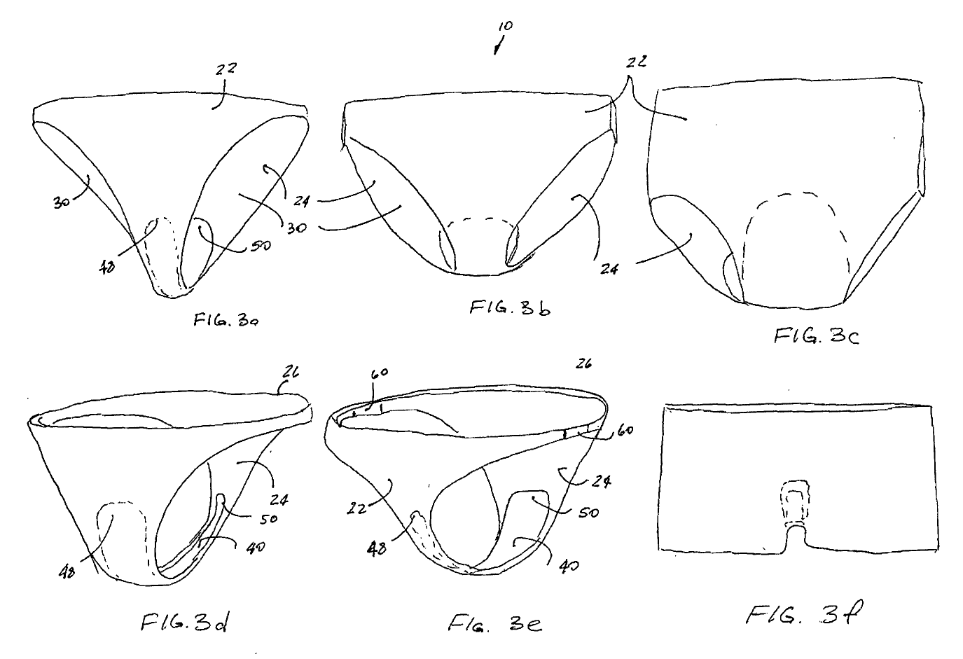

An elongated absorption pad 40 is overlaid over and secured to the crotch part 28 and the lower portion of each front and rear part 22 and 24 respectively. The absorption pad 40 may be manufactured according to any of the methods presently employed in manufacturing sanitary napkins, tampons, diapers and the like articles and therefore its detail description is omitted for the sake of brevity. It will be understood that the absorption pad 40 may be manufactured to provide various degrees of absorbing protection characterized by light flow or heavy flow of menses. In a conventional manner, as shown in FIG. 4, the absorption pad 40 has a fluid-permeable human body facing layer 42, a fluid-impermeable panty body engaging layer 44 and an absorbent layer 46 disposed intermediate the fluid-permeable and the fluid-impermeable layers 44 and 46 respectively. The absorption pad 40 is secured to the interior surface of the panty body 20 with any conventional securing method, for example such as stitches 54 disposed along its rim 52.

The absorption pad 40 has a predetermined length. It is presently preferred, in order to increase absorption of menses over a conventionally available sanitary napkins and tampons, to position a front end 48 of the absorption pad 40 above external genital organs of a wearer and to position a rear end 50 thereof in close proximity to an anus of the wearer. This increases the effective absorption area provided by the absorbent layer 46 and enables the absorption pad 40 of the present invention to absorb increases in menstrual flow over conventionally available sanitary napkins and tampons. It is presently preferred for such predetermined length to be about 12.0 inches or greater.

It has been found necessary, for preventing undesirable protrusion of the absorption pad 40 beyond the boundary of the panty body 20, to size the absorption pad 40 so as to contain the rim 52 thereof within such panty body boundary, particularly in the area of the crotch part 28. Accordingly, the side edges 56 of the absorption pad 40 taper inwardly toward one end thereof. As the front part 22 and the rear part 24 differ for many styles/cuts of the panty body 20, the direction of the taper varies with such style. By way of an example of FIGS. 3a and 3e, when the front part 22 is narrower than the rear part 24, the side edges 56 taper toward the front end 48 of the absorption pad 40. Buy way an example of FIG. 3d, when the rear part 24 is narrower than the front part 22, the side edges 56 taper toward the rear end 50 of the absorption pad 40. It will be understood, that the absorption pad 40 may be provided in a conical, frustoconical, triangular, hour-glass, or teardrop shape. Thus, the wearer of the undergarment 10 is comforted by both absorption of menses and by aesthetics of hidden absorption pad 40 when such wearer is required to disrobe, particularly, when the panty body 20 has a reduced coverage area due to a tapered front part 22 or rear part 24, as depicted in FIGS. 3a, 3d and 3e, respectively, forming a narrow and varying width crotch part 28.

It is within the scope of the present invention to provide a pair of release means 60 each for detachably securing the front part 22 and the rear part 24 at one side and the top edge 26 of the panty body 20. Such release means 60 includes but not limited to loop and a hook fastener, strings snaps, buttons, adhesive and various combinations thereof, presently employed in fastening portions of undergarments together. Thus the wearer is advantaged by easy pull-on-pull-off effort minimizing unintentional contact between soiled surface and the article of clothing or minimizing leakage from the heavy saturated absorption pad 40. The soiled undergarment 10 is simply wrapped in itself and then secured by the release means 60 for easy and private disposal.

It is also within the scope of the present invention to provide a carrying case or a pouch 70. It has been found advantageous to form such carrying case or pouch 70 from a material sufficient to prevent leakage from a soiled undergarment 10 enclosed therewithin. For example, such material can be a plastic. It is further contemplated to provide a flexible plastic material in order to minimize storage space inside the pocket or a purse. Thus, the wearer is enabled to temporarily store or keep the soiled undergarment 10 without staining the storage environment until proper disposal is available. Advantageously, the color of and/or graphics on an exterior surface of the carrying case or pouch 70 formed to respectively match color and graphics of the panty body 20.

It will be appreciated that the combined disposable panty and sanitary pad undergarment 10 offers women an advantage of the sanitary pad 40 which is affixed to the interior surface of the panty body 20 and will not move or shift. It would be appreciated that the wearer remains comfortable and fully protected during menstrual cycle in a clean and convenient manner. Thus, the present invention overcomes the disadvantages of prior art conventional sanitary pad which is simply placed into a panty or temporarily secured and which is generally will move or shift during use. The present invention also offers increased absorption of menses in combination with a reduced coverage panty body style and with hidden absorption pad 40 when the user wears such undergarment 10. The present invention also contemplates to manufacture the panty body 20 from an absorbent material, for example such as cotton, cotton-blend or synthetic breathable materials, for additional protection, should the leakage occur from the pad 40. The undergarment 10 of the present invention can be also manufactured in various styles, sizes, designs and colors, offering an advantage over presently available diaper like prior art undergarments. The user is also unburdened by incorrect placement of adhesive associated with conventional sanitary feminine products thus preventing damages to the sanitary product or harm to delicate skin.

Although the present invention has been shown in terms of the combination of panty body 20 and the absorption pad 40, it will be apparent to those skilled in the art, that the present invention may be used with additional sanitary feminine products for increased protection.

Thus, the present invention has been described in such full, clear, concise and exact terms as to enable any person skilled in the art to which it pertains to make and use the same. It will be understood that variations, modifications, equivalents and substitutions for components of the specifically described embodiments of the invention may be made by those skilled in the art without departing from the spirit and scope of the invention as set forth in the appended claims.

I claim:

1. A combined disposable panty and sanitary pad undergarment, said undergarment comprising:
(a) a panty body having a front part, a rear part forming in combination with said front part a top edge of said panty body, and a crotch part joining lower portions of each of said rear and front part and forming a pair of leg openings; and
(b) an absorption pad overlaid over and secured to said crotch part and said lower portion of said each front and rear part, said absorption pad having a fluid-permeable human body facing layer, a fluid-impermeable panty body engaging layer and an absorbent layer disposed intermediate said fluid-permeable and said fluid-impermeable layers, said absorption pad having a predetermined length whereby a front end thereof is positioned above external genital organs of a wearer and whereby a rear end thereof is positioned in close proximity to an anus.

2. The undergarment, according to claim 1, wherein a width of said absorption pad is sized so as to contain a rim thereof within a boundary of said panty body.

3. The undergarment, according to claim 1, wherein side edges of said absorption pad at least partially taper inwardly toward one end thereof.

4. The undergarment, according to claim 3, wherein said side edges at least partially taper toward said rear end of said absorption pad.

5. The undergarment, according to claim 3, wherein said side edges at least partially taper toward said front end of said absorption pad.

6. The undergarment, according to claim 1, wherein said undergarment includes a pair of release means each for detachably securing said front part and said rear part at one side of said top edge of said panty body.

7. The undergarment, according to claim 6, wherein said release means includes one of loop and hook fastener, strings snaps, buttons, adhesive and various combinations thereof.

8. The undergarment, according to claim 1, wherein said undergarment includes a carrying case or a pouch formed from a material sufficient to prevent leakage from or strike through a soiled undergarment enclosed therewithin.

9. A combined disposable panty and sanitary pad undergarment, said undergarment comprising:
(a) a panty body having a front part, a rear part forming in combination with said front part a top edge of said panty body, and a crotch part joining lower portions of each of said rear and front part and forming a pair of leg openings; and
(b) an absorption pad overlaid over and secured to said crotch part and said lower portion of said each front and rear part, said absorption pad having a fluid-permeable human body facing layer, a fluid-impermeable panty body engaging layer and an absorbent layer disposed intermediate said fluid-permeable and said fluid-impermeable layers, said absorption pad having a predetermined shape wherein side edges thereof at least partially taper inwardly toward one end thereof.

10. A combined disposable panty and sanitary pad undergarment, said undergarment comprising:
(a) a panty body having a front part, a rear part forming in combination with said front part a top edge of said panty body, and a crotch part joining lower portions of each of said rear and front part and forming a pair of leg openings, said panty body manufactured from an absorbent material;
(b) an absorption pad overlaid over and secured to said crotch part and said lower portion of said each front and rear part, said absorption pad having:
(i) a fluid-permeable human body facing layer,
(ii) a fluid-impermeable panty body engaging layer,
(iii) an absorbent layer disposed intermediate said fluid-permeable and said fluid-impermeable layers,
(iv) wherein said absorption pad having a predetermined length whereby a front end thereof is positioned above external genital organs of a wearer and whereby a rear end thereof is positioned in close proximity to an anus, and
(v) wherein side edges of said absorption pad at least partially taper toward a predetermined end thereof, and
(vi) wherein, a width of said absorption pad is sized for containment within a boundary of said panty body; and
(c) a pair of release means each for detachably securing said front part and said rear part at one side of said top edge of said panty body.
